(19) United States
(12) Patent Application Publication
(10) Pub. No.: US 2006/0093679 A1
(43) Pub. Date: May 4, 2006

(54) Fast releasing, solid administration form for oral application of active ingredients which are hard to dissolve

(76) Inventors: Jorg Mayer, Berlin (DE); Karsten Cremer, Berlin (DE); Lutz Krohne, Berlin (DE)

Correspondence Address: Moser Patterson & Sheridan;Zimmermann & Partner, Patent Counsel Suite 1500, 3040 Post Oak Boulevard, Houston, TX, 77056, US

(21) Appl. No.: 10/529,284

(22) PCT Filed: Sep. 24, 2003
(86) PCT No.: PCT/EP03/10630

§ 371 Date: Aug. 18, 2005

(30) Foreign Application Priority Data
Sep. 25, 2002 (DE) 10244504.4

(51) Int. Cl. A61K 9/50; A61K 9/16

(52) U.S. Cl. 424/490

(57) ABSTRACT

The present invention is concerned with solid, single-dose dosage forms for an increased rate of release of slightly soluble active ingredients. The dosage forms are based on a coherent matrix which disintegrates rapidly in physiological fluids. The matrix comprises one or more slightly soluble active ingredients in the form of fast-release micro- or nanocapsules. The dosage form is particularly suitable for administering those slightly soluble active ingredients for which a rapid onset of action is desired.

The invention is concerned with solid administration forms for oral administration of active ingredients. It relates in particular to matrix-like, single-dose administration forms intended for rapid disintegration in the oral cavity, to the production thereof and to the use thereof for producing medicaments.

Administration forms for administering systemically active active ingredients are preferably taken orally by most people. A number of administration forms are available for this purpose, for example conventional tablets with or without film, chewable tablets, suckable tablets, soft capsules, hard capsules, drops, liquids or instant granules. Particularly common administration forms are tablets and soft or hard capsules for swallowing. Children and elderly people who find it difficult to swallow tablets or capsules often by contrast receive drops or liquids.

In the past, alternative dosage forms which are as easy to swallow as liquid preparations but at the same time have the most important advantages of solid, single-dose pharmaceutical forms—for example accurately predetermined dosage or increased stability of the active ingredient in dry form—have been developed for patients who swallow tablets or capsules unwillingly or not at all. These are called orally disintegrating dosage forms, which can in principle be assigned to three different types. A first group was developed on the basis of conventional tablet technology. Tablets which disintegrate in the mouth within a short time without chewing are usually more porous than customary tablets, are compressed with a lower pressure, but comprise a high proportion of the cohesion-conferring binder so that, despite the porosity, a certain mechanical stability can be achieved. A second group are the oral lyophilisates which are produced as highly porous matrices by freeze-drying a solution or suspension of active ingredient and suitable excipients. The third group is formed by the rapidly disintegrating film-like preparations which have recently become available on the market as mouth cosmetic (Listerine PocketPak, Pfizer) and are also proposed for the administration of active pharmaceutical ingredients.

These dosage forms, which disintegrate very rapidly in the mouth, are usually regarded as particularly suitable for the administration of active ingredients to control acute diseases or conditions such as migraine. This is on the one hand also correct since the patient can, after all, administer the medication immediately when the symptoms first appear, unlike the case of conventional tablets or capsules, for which a suitable situation and the availability of drinking water must be awaited for intake thereof. However, on the other hand, immediate intake and rapid disintegration of the pharmaceutical form are not associated with a fast onset of action. The step which is usually rate-determining for the onset of action of a medication is dissolution of the active ingredient in the gastric juice, or saliva or intestinal juice. This may take a rather long time especially with slightly soluble active ingredients. It is by no means complete when a dosage form has disintegrated; on the contrary, it has where appropriate only just started. However, since dissolution of the active ingredient is the precondition for absorption and the onset of action, a fast onset of action after administration is scarcely possible without speeding up thereof in the case of a slightly soluble active ingredient.

There is thus a need for dosage forms which enable active ingredient dissolution to be speeded up and thus lead to a fast onset of action. There is a particular need for orally disintegrating dosage forms with a fast onset of action.

It is an object of the invention to provide such a dosage form.

The object is achieved by a solid dosage form for oral administration comprising a coherent matrix with a disintegration time of less than 2 minutes, in which the matrix comprises an active ingredient which is slightly soluble in a physiological fluid and which is in the form of fast-release micro- or nanocapsules

For the purposes of the invention, a dosage form means a preparation for administering an active ingredient. A dosage form ordinarily comprises one or more suitable excipients besides the active ingredient itself. Solid dosage forms include inter alia tablets, film-coated tablets, sublingual tablets, buccal tablets, oral lyophilisates and oral films.

An active ingredient may be for example a pharmaceutical or therapeutic active ingredient, a mixture of active ingredients, a diagnostic substance, a vitamin, vital substance, nutrient or a mineral. The active ingredient is preferably a therapeutic active ingredient or a mixture of therapeutic active ingredients. Since the invention aims to achieve a fast release of the active ingredient, the dosage form is especially suited in particular for active ingredients which are employed for the treatment of an acute disorder or symptoms which occur acutely. Examples of such classes of active ingredients are analgesics, migraine remedies, spasmolytics, antiemetics, antiallergics, antidiarrheals, antihypertensives, antihypotensives, antivertigo agents, analeptics, psychoactive drugs, antidotes, habit cessation aids, antiarrhythmics, sedatives, hypnotics, antiepileptics, tocolytics, diagnostics or substances to counter erectile dysfunction.

Besides the slightly soluble active ingredient, the dosage form may also comprise a second active ingredient which is either slightly soluble or soluble. The second active ingredient may, if it is slightly soluble and would, just like the first, profit from speeded up dissolution after administration, likewise be in micro- or nanoencapsulated form. If, on the other hand, it is water-soluble, it will advantageously be in unencapsulated form.

The fact that the dosage form has the structure of a coherent matrix means that it is not a disperse form such as, for example, a powder or granules, but is a solid, shaped “single unit” which comprises in each case one dose unit and in which the active ingredient is dispersed in a carrier matrix. The matrix may optionally be coated with a saliva-soluble film.

Matrix-like solid dosage forms with rapid disintegration on contact with aqueous liquids are usually produced either as highly porous shaped articles by tableting or freeze-drying or as oral films by coating and drying—alternatively by extrusion. A detailed description of these dosage forms, their production and functionality is to be found in K. Cremer: Orally Disintegrating Dosage Forms, Berlin 2001, the contents of which are expressly incorporated herein by reference. Besides the active ingredient, a proportion of one or more physiologically acceptable excipients is almost always necessary to construct a matrix. Oral films comprise in this connection in particular water-soluble, film-forming polymers such as gelatin, polyvinyl alcohol, polyvinylpyrrolidone, pullulan, hydroxypropylcellulose, hydroxypropylmethylcellulose and the like. In tableted matrices there is ordinarily use in particular of a binder, preferably a readily plastically deformable dry binder such as microcrystalline cellulose. Lyophilized shaped articles may comprise as matrix formers or structuring agents a polymer such as, for example, gelatin, and in addition frequently a sugar alcohol such as mannitol. Further pharmaceutical excipients may be used if required, e.g binders, thickeners, surfactants, wetting agents, stabilizers, antioxidants, aromas, taste improvers, sweeteners, fillers, absorption improvers, colorants, pigments, plasticizers, lubricants, release agents, flow regulators, etc.

The disintegration time of the matrix should be less than 2 minutes. In this connection, the disintegration time refers to the disintegration time measured in vivo on oral use, which should be determined without chewing the dosage form, or which is measured in vitro by the method of a recognized pharmacopoeia (e.g. the United States Pharmacopoeia 25) under standard conditions, the disintegration medium which should be used in accordance with the purpose of use not being simulated gastric fluid but water or physiological buffer solution with a pH of >5.5. Depending on the configuration of the matrix it is perfectly possible for considerably shorter disintegration times to be achieved—especially in the case of freeze-dried matrices. A preferred matrix according to the invention has a disintegration time of less than 1 minute. A particularly preferred disintegration time in the case of tableted and film-like matrices is less than 30 seconds. In the case of lyophilized matrices, a disintegration time of less than 20 seconds is preferred. In a further configuration, a lyophilized matrix according to the invention has a disintegration time of less than 10 seconds, in particular of less than 5 seconds.

The subject matter of the invention is further defined by the matrix comprising an active ingredient which is slightly soluble in a physiological fluid and which is in the form of fast-release micro- or nanocapsules. An active ingredient is slightly soluble according to the invention if the active ingredient has a solubility in a physiological fluid which is relevant for release, i.e. in saliva, simulated saliva, gastric juice, simulated gastric fluid, small intestinal juice or simulated small intestinal fluid, not exceeding about 1% (w/v). In the sense of an exception from this definition it is possible to use a further criterion of slight solubility which is essential for use of the invention, which is that it applies when the amount of active ingredients present in the matrix does not dissolve in about 500 ml of physiological fluid at 37° C. In a preferred embodiment, 200 ml of physiological fluid are insufficient to dissolve the amount of active ingredient present. Particularly preferred active ingredients are those which do not dissolve completely in 100 ml or even 50 ml of physiological fluid. It is precisely with such active ingredients that the dosage form of the invention is intended to enable rapid release which cannot be achieved solely through the use of a rapidly disintegrating matrix as active ingredient carrier.

The micro- or nanoencapsulated state in which the slightly soluble active ingredient is dispersed in the matrix is at the heart of the solution to the problem: the active ingredient is present according to the invention in the form of fast-release micro- or nano-capsules. In this connection, the terms “microcapsules” and “nanocapsules” refer to all known types of micro- and nanoparticles having a capsule-like structure, i.e. they comprise a core and a shell which can be distinguished from the core. Whereas the core comprises the predominant part of the slightly soluble active ingredient and in addition, if required, one or more pharmaceutical excipients, the shell may be composed of various materials, e.g. consist of one or more polymers, of a mixture of polymeric and nonpolymeric substances, of lipids and lipid-like substances, or of a combination of lipids and polymers. To this extent, the term micro- or nanocapsules also include liposomes.

The fact that the micro- or nanocapsules are intended to be fast-release species means that the capsule shells have a high permeability for dissolved molecules of the active ingredient present and a negligible release-slowing effect. The permeability is high for example when the capsules are able to release the slightly soluble active ingredient present therein within 60 minutes under sink conditions at 37° C. Preferred capsules are those which release their active ingredient in one of the physiological fluids defined above within fewer than 30 minutes under the stated conditions. Particularly preferred capsules are those whose release takes place within 15 minutes, in particular within 10 minutes. In some cases it will even be possible to achieve release times of less than 5 or less than 2 minutes, specifically when the capsule shell is particularly permeable for the active ingredient, and the particle size of the capsule is particularly small, as will be explained further below. For the purposes of the invention, release has taken place when at least about 90% by weight of the active ingredient present in the capsules or in the matrix or else the dosage form is in dissolved form in the release medium. The release time from the capsules will be determined best by a recognized in vitro release test (e.g. according to United States Pharmacopoeia 25), although the apparatus and the conditions must be chosen so that the required sink conditions are maintained. This may mean that a flow cell must be used, instead of the most frequently used paddle apparatus, for certain active ingredients. Further preferred capsules are those which release the active ingredient in the stated periods not only in the acidic gastric juice but also in saliva, which tends to be neutral.

The fast-release micro- and nanocapsules contrast with active ingredient-containing capsules which mainly serve for taste masking, and which are therefore aimed in particular at not releasing an active ingredient at least in the saliva in particular, because otherwise no taste masking would take place. Capsules of this type have already been described as constituent of dosage forms which show rapid oral disintegration, see, for example, U.S. Pat. No. 5,607,697 and WO 98/14179. Capsules which can be employed for this purpose considerably slow the release of the active ingredient in saliva. The capsules usually have relatively thick walls and tend to show low permeability in a neutral medium and have a diameter of more than 10 μm.

By contrast, the capsules of the invention tend to have thin walls, with typical average wall thicknesses of less than about 20 nm, and in some cases of less than about 10 nm. They typically have an average diameter not exceeding about 10 μm, measured as z average by quasielastic light scattering or photon correlation spectroscopy. Capsules with an average diameter of about 200 nm to 5 μm are preferred, particularly those with an average diameter of about 400 nm to 3 μm, and very particularly those with an average diameter of about 500 nm to 2 μm. Particle sizes as small as these mean that the surface area is particularly large, and thus the rate of dissolution of the slightly soluble active ingredient may be very considerably increased. In fact, a considerable advantage of the invention is the creation of a possibility for incorporating the active ingredient in a state with a particularly large surface area into a rapidly disintegrating matrix. Otherwise, the provision of such a large surface area is extremely difficult. Although slightly soluble active ingredients can often be reduced to an average particle size of from 5 to 10 μm by complicated micronization, below this range the resultant surface energies are so large that the relevant particles are highly prone to agglomeration and can scarcely be handled.

Capsules in the preferred size range can—as mentioned above—be composed of various materials in terms of their shell material, e.g. consist of one or more polymers, of a mixture of polymeric and nonpolymeric substances, of lipids and lipid-like substances, or of a combination of lipids and polymers. In order to achieve high permeability, in particular hydrophilic materials which are water-soluble or swellable in water should be employed, with water-soluble capsules being more suitable for tableted matrices, while hydrophilic, swellable but insoluble materials are likely to be more suitable for lyophilisates and films. Stable but highly permeable capsule shells can be prepared for example with a polyelectrolyte and a multiply charged low molecular weight counter ion. Suitable capsules can likewise be produced by complex formation from at least two opposing polyelectrolytes. Polycations which can be employed to produce polyelectrolyte complex capsules include inter alia polyvinylamines, polyvinylpyridines, polyallylamines, polyethyleneimines, ammonium salts of polyacrylates, aminated dextrans, aminated celluloses, aminated pectins, chitosan, polylysine, spermine, but also corresponding copolymers. Polyanions which can be employed include inter alia polyacrylic acid, polyvinylsulfonic acids, polyvinylphosphonic acids, polyphosphoric acids, polymaleic acids, polystyrenesulfonic acids, polylactic acids, polyglycolic acids, carboxymethylcelluloses, carboxymethyldextrans, hyaluronic acid, chitosan sulfates, cellulose sulfates, sulfoethylcelluloses, chondroitin sulfates, dextran sulfates, carrageenans, pectins, gum arabic, lignin sulfates, nucleic acids, alginic acids, but also corresponding copolymers. Multiply charged lower molecular weight ions with which slightly soluble salts can be formed with a suitable polyelectrolyte include inter alia yttrium(III) cations, terbium(III) cations, iron(III) cations.

Thin-walled micro- and nanocapsules based on polyelectrolyte complexes or slightly soluble salts of polyelectrolytes can be produced by various known processes, e.g. by coacervation, spray drying, conventional double emulsion processes. However, a particularly suitable process is a so-called layer-by-layer process (LBL) by which such capsules can be constructed by layered adsorption or electrostatic self-assembly of polyelectrolytes on nano- or micro-disperse surfaces. The process is described in detail in the following documents, the contents of which are expressly incorporated herein by reference: WO 99/47252, WO 99/47253, WO 00/03797, WO 00/77281 and WO 01/51196. Capsules produced by this process may have a wall thickness of less than 20 nm or even of less than about 10 nm. If the polyelectrolytes are selected suitably, such thin-walled capsules simultaneously have such a high permeability for lower molecule weight substances that diffusion thereof through the capsule wall takes a few minutes at the most, but usually only a few seconds, so that the capsules comply in an outstanding manner with the requirements of the invention concerning rapid release.

A particularly rapid release of a slightly soluble active ingredient from a dosage form of the invention occurs especially when the time required for the active ingredient to pass through the capsule shell is considerably less than the time gained, owing to the high degree of dispersity, on dissolution of the active ingredient.

One alternative to the polyelectrolyte-containing capsules is represented by capsules with shells of lipid layers or lipid bilayers, which are also known as liposomes, this term referring in particular to the state of such capsules in liquid dispersion. Lipid layers or lipid bilayers are also able to stabilize a micro- or nanodisperse, slightly soluble active ingredient with negligible hindrance to its diffusion out of the capsule. This applies in particular to layers based on phospholipids, have a low phase transition temperature and thus are in fluid form at body temperature, e.g. phospholipids having at least one unsaturated fatty acid residue or those having short to medium chain fatty acid residues such as, for example, dipalmitoylphosphatidylcholine. Combination of lipid layers and polyelectrolyte layers for constructing micro- and nanocapsules is also known and is described in detail in the documents DE 101 09 898, DE 100 43 011, DE 100 10 264, DE 199 54 843 and DE 198 52 928.

As already mentioned above, the rapidly disintegrating matrix can be produced in the form of a tablet. For this purpose, the micro- or nanocapsules comprising the slightly soluble active ingredient are processed with the further excipients required to construct the matrix and form the tablet, by a process known in pharmaceutical technology, to a powder mixture or to granules. An alternative possibility is to produce granules from excipients for subsequent admixture of the capsules. This is followed by compression of the granules or the powder mixture with a low to moderate compressive force to give tablets of suitable shape and relatively high porosity. Since tablets with high porosity typically have a lower friability than usual tablets, not every tablet shape is equally suitable; on the contrary, shapes with pronounced convexities and facets are to be preferred. It may where appropriate be advantageous for the tablet to be subsequently treated by sintering. For a sufficiently stable shaped article to be used at all under low compressive forces, certain formulation and process parameters must be complied with, but are easily accessible to the skilled worker. Reference may be made in particular to the documents WO 01/10418, WO 99/44580, WO 99/04758, WO 98/14179, WO 00/09090, EP 548 356, WO 99/04763, WO 00/27357 and WO 00/51568.

If the matrix is to have a film-like configuration so that rapid disintegration is brought about in particular through a large outer—and not as for the tablets through a large inner—surface area, it is likewise possible to proceed according to known formulation and process principles, which need to be varied so that the micro- or nanocapsules described hereinbefore are to be incorporated. For this purpose, it is advisable to dissolve, disperse and, where appropriate, homogenize the polymeric film former(s) and the further excipients necessary to construct the matrix in a suitable solvent to give a spreadable composition. The solvent or mixture thereof should be selected so that the slightly soluble active ingredient present in the capsules is only very slightly soluble, or if possible insoluble, therein. The capsules are then admixed with the composition. The composition is then applied to a suitable support and dried, preferably using heat. The individual dose units are subsequently divided off by cutting or punching. Formulation and process parameters which must additionally be complied with are to be found inter alia in the documents EP 259 749, DE 40 18 247, DE 44 19 824, DE 196 52 268, DE 196 52 188, DE 198 00 682, DE 198 06 966, DE 198 37 073, DE 196 46 392 and DE 198 56 845.

If the matrix is to be configured as lyophilized shaped article, it is likewise possible to use known excipients to construct it, and to proceed by known processes. The fluid or highly viscous composition for producing the lyophilized shaped article comprises a solvent and a gelatin-containing carrier material into which the nano- or microcapsules described hereinbefore are to be incorporated. The carrier must be soluble in the chosen solvent, and the solvent must be inert towards the pharmaceutically active substance.

Water is preferably used to produce said composition, and is frozen and subsequently sublimed. Deonized water will usually be preferred for the production.

Carrier material means the abovementioned thickeners which are blended with the dosage form and provide a solid matrix to support the encapsulated active ingredients after the solvent has been removed by sublimation. The or the encapsulated pharmaceutically active substances are incorporated into the matrix of the carrier material. Examples of gelatins suitable as carrier material include simple gelatin, partially hydrolyzed gelatin, and succinylated gelatin. The carrier matrix may be further supplemented by excipients from the group of cryoprotectants in order to achieve amorphous freezing of the solvent and protection of the also frozen nano- or microcapsules. If the chosen solvent is water, examples of these excipients are mannitol, sucrose, glucose or the like

The production of the fluid or highly viscous composition of the lyophilized dosage form of the invention is typically prepared in a relatively large batch and divided up into small controlled dosage quantities by introducing the composition into one or more recesses in a shaped dish or the like. The recess will generally correspond to the shape and size of the finished dosage form. A plurality of these recesses will normally be formed in one piece of a sheet-like material. This sheet-like material may for example be composed of thermoplastic material with recesses shaped under the influence of heat.

The fluid or highly viscous composition can be introduced into the wells by known methods. Likewise, the freezing takes place by a prior art method which is suitable for producing a sublimable frozen item. The freezing apparatus is operated at a temperature which is low enough to solidify the mixture completely.

The frozen quality of solvent in the composition is subsequently sublimed. The sublimation should preferably be carried out in a freeze dryer in which the now frozen composition in the recesses is exposed to a reduced pressure. The sublimation can be assisted by the controlled input of heat. For this purpose, the temperature of the supporting surfaces on which the frozen mixture is located can be raised in order to speed up the sublimation process. After the sublimation is complete, the freeze dryer is returned to atmospheric pressure level, and the now solid shaped articles are taken out of the freeze dryer. The material comprising the solid shaped articles is then usually sealed with a suitable sheet which is applied either by bonding or with heat sealing over the recesses comprising the shaped articles.

In one of the preferred embodiments, gelatin and mannitol are employed as carrier materials. Whereas gelatin is ordinarily employed in excess in the prior art, it surprisingly proves to be particularly advantageous for lyophilized matrices with polyelectrolyte-coated active ingredient particles if the ratio is shifted distinctly in favor of mannitol. A gelatin-mannitol ratio of about 1:1 to 1:3 is particularly preferred, for example one of 1:2.

Further detailed information on possible configurations of lyophilisates for oral use and on the production thereof are known to the skilled worker. Reference may be made in particular to the patents U.S. Pat. No. 4,642,903, U.S. Pat. No 4,754,597, U.S. Pat No. 4,758,598 and U.S. Pat No. 5,188,825.

### EXAMPLE 1

Preparation of Formulation with 25 mg of Ketoprofen

In the first production step, the micronized active ingredient is encapsulated. This takes place by means of LBL technology as disclosed in the patent applications WO 01/51196 and WO 99/47252. The micronized active ingredient is disclosed in an aqueous sodium dodecyl sulfate solution and encapsulated with the polyelectrolytes gelatin A and chondroitin sulfate under acidic conditions. Gelatin and mannitol are dissolved in water, the pH is adjusted to 3.5, and the ketoprofen suspension is added to this solution and homogenized. The preparation of 25 mg units takes place by charging suitable preshaped blister cards, introducing 490 mg of the dispersion into each single recess (12 mm diameter). The product is subsequently frozen at −80° C. and freeze-dried as, for example, in WO 95/01782.

| | | % by weight of |
|---|---|---|
| Ingredient | Weight (mg) | the composition |
| Purified water EP/USP* | 441.48 | 90.10 |
| LBL coated ketoprofen | 25.00 | 5.10 |
| Gelatin | 7.84 | 1.60 |
| Mannitol EP/USP | 15.68 | 3.20 |
| HCl | qs pH 3.5 | qs pH 3.5 |
| Total | 490 | 100 |

*means removed during the freeze-drying process

It is possible further to add excipients such as, for example, aspartame and flavorings to the formula.

Active Ingredient Release

The release in vitro was determined using a release apparatus as specified in the Pharmacopoea Europaea using the paddle method at 50 revolutions per minute in 600 ml of 0.1N HCl at 37° C. and with detection by UV spectrometry, and is shown in the table below. All the samples were passed through a 0.45 μm filter. It should be noted that non-sink conditions in an acidic medium were used. If a test is carried out under sink conditions as specified in the Pharmacopoea Europaea, it is to be expected that this would lead an increased rate of release.

| | Total amount of the active |
|---|---|
| Time [min] | ingredient released [%] |
| 0 | 0 |
| 2 | 53 |
| 6 | 74 |
| 10 | 79 |
| 14 | 76 |
| 20 | 78 |
| 30 | 80 |
| 45 | 76 |
| 60 | 83 |

### EXAMPLE 2

Preparation of Formulation with 25 mg of Carbamazepine

In the first production step, the micronized active ingredient is encapsulated. This takes place by means of LBL technology as disclosed in the patent applications WO 01/51196 and WO 99/47252. The micronized active ingredient is disclosed in an aqueous sodium dodecyl sulfate solution and encapsulated with polyelectrolytes gelatin A and carboxymethyl-cellulose Gelatin and mannitol are dissolved in water and the carbamazepine suspension is added to the solution and homogenized. The preparation of 25 mg units takes place by charging suitable preshaped blister cards, introducing 500 mg of the dispersion into each single recess (12 mm diameter). The product is subsequently frozen at −80° C. and freeze-dried as, for example, in WO 95/01782.

| | | % by weight of |
|---|---|---|
| Ingredient | Weight (mg) | the composition |
| Purified water EP/USP* | 451 | 90.2 |
| LBL coated | 25 | 5 |
| carbamazepine | | |
| Gelatin | 8 | 1.6 |
| Mannitol EP/USP | 16 | 3.2 |
| Total | 500 | 100.0 |

*means removed during the freeze-drying process

It is possible further to add excipients such as, for example, aspartame and flavorings to the formula.

Active Ingredient Release

The release in vitro was determined using the release Pharmacopoea Europaea paddle method at 50 rpm in 800 ml of 0.1N HCl at 37° C. and with detection by UV spectrometry, and is shown in the table below. All the samples were passed through a 0.45 μm filter.

| | Total amount of the active |
|---|---|
| Time [min] | ingredient released [%] |
| 0 | 0 |
| 5 | 15 |
| 10 | 24 |
| 15 | 55 |
| 20 | 65 |
| 25 | 72 |
| 30 | 75 |
| 45 | 80 |
| 60 | 85 |
| 90 | 90 |
| 120 | 90 |

In a comparative test under identical conditions, the release of the same active ingredient from Tegretol tablets was 51% after 60 minutes and 68% after 120 minutes.

1. A solid dosage form for oral administration comprising a coherent matrix with a disintegration time of less than 2 minutes, wherein:
the matrix comprises an active ingredient which is slightly soluble in a physiological fluid and which is in the form of fast-release capsules selected from at least one of micro- and nanocapsules,
the capsules comprising a core and a shell,
the core comprising the slightly soluble active ingredient,
the shell consists essentially of a material with high permeability for the slightly soluble active ingredient, and
the shell of the capsules comprising a complex of at least one polyelectrolyte and a counter ion to the polyelectrolyte.

2. The dosage form as claimed in claim 1, characterized in that the matrix has a disintegration time of less than 30 seconds.

3. The dosage form as claimed in claim 1, characterized in that release of the active ingredient is virtually complete within 30 minutes.

4. The dosage form as claimed in claim 1, characterized in that the matrix further comprises gelatin and mannitol in a ratio of 1:1 to 1:3.

5. The dosage form as claimed in claim 1, characterized in that the slightly soluble active ingredient is selected from at least one of an analgesic, a migraine remedy, a spasmolytic, an antiemetic, an antiallergic, an antidiarrheal, an antihypertensive, an antihypotensive, an antivertigo agent, a psychoactive drug, an antidote, habit cessation aid, an antiarrhythmic, a sedative, a hypnotic, a tocolytic, a diagnostic and a substance to counter erectile dysfunction.

6. The dosage form as claimed in claim 1, characterized in that the capsules have an average particle size of not more than about 10 μm.

7. The dosage form as claimed in claim 1, characterized in that the counter ion is a polyelectrolyte.

8. The dosage form as claimed in claim 1, characterized in that the capsules are produced by layered electrostatic self-assembly.

9. The dosage form as claimed in claim 1, characterized in that the shell of the capsules comprises a material selected from at least one of a lipid layer and a lipid bilayer.

10. The dosage form as claimed in claim 1, characterized in that the matrix is produced by compressing a material selected from at least one of powder and granules.

11. The dosage form as claimed in claim 1, characterized in that the matrix is produced by freeze-drying a substance selected from at least one of a fluid and a highly viscous composition.

12. The dosage form as claimed in claim 1, characterized in that the matrix is produced by solidifying a composition which has been spread out into a film.

13-16. (canceled)

17. The dosage form as claimed in claim 4, wherein the capsules have an average size of less than about 10 μm.

18. A method of producing a solid dosage form for oral administration that comprises a coherent matrix with a disintegration time of less than two minutes, comprising:
providing an active ingredient which is slightly soluble in a physiological fluid and which is in the form of fast-release capsules selected from at least one of micro- and nanocapsules, wherein the capsules comprise a core comprising the slightly soluble active ingredient and a shell consisting essentially of a material with high permeability for the slightly soluble active ingredient, the shell of the capsules comprising a complex of at least one polyelectrolyte and a counter ion to the polyelectrolyte;
mixing the capsules with matrix-forming, physiologically acceptable excipients to provide a mixture; and
forming the mixture into dose units.

19. The method of claim 18, wherein forming the mixture into dose units includes compressing the mixture into tablets.

20. The method of claim 18, further comprising mixing the mixture with a liquid carrier to provide a solution, wherein forming the mixture into dose units includes dividing and freeze-drying the solution.

21. The method of claim 18, further comprising mixing the mixture with a liquid carrier to provide a solution, wherein forming the mixture into dose units includes spreading the solution into a film and drying the film.

22. The method of claim 18, wherein the capsules have an average particle size less than about 10 μm.

23. The method of claim 18, wherein the active ingredient is a therapeutic.

24. A method of producing a medicament for the treatment of acute diseases, comprising:
forming a coherent matrix with a disintegration time of less than two minutes, wherein the matrix comprises an active ingredient which is slightly soluble in a physiological fluid and which is in the form of fast-release capsules having an average size of less than about 10 μm, wherein the capsules comprise a core comprising the active ingredient and a shell consisting essentially of a material with high permeability for the active ingredient, the shell of the capsules comprising a complex of at least one polyelectrolyte and a counter ion to the polyelectrolyte.

25. The dosage form as claimed in claim 4, characterized in that the slightly soluble active ingredient is selected from at least one of an analgesic, a migraine remedy, a spasmolytic, an antiemetic, an antiallergic, an antidiarrheal, an antihypertensive, an antihypotensive, an antivertigo agent, a psychoactive drug, an antidote, habit cessation aid, an antiarrhythmic, a sedative, a hypnotic, a tocolytic, a diagnostic and a substance to counter erectile dysfunction.

26. The dosage form as claimed in claim 25, wherein the capsules have an average size of less than about 10 μm.

27. The dosage form as claimed in claim 5, wherein the capsules have an average size of less than about 10 μm.

28. The dosage form as claimed in claim 4, characterized in that the shell of the capsules comprises a material selected from at least one of a lipid layer and a lipid bilayer.

29. The dosage form as claimed in claim 5, characterized in that the shell of the capsules comprises a material selected from at least one of a lipid layer and a lipid bilayer
